(19) United States
(12) Patent Application Publication
(10) Pub. No.: US 2026/0226391 A1
(43) Pub. Date: Aug. 6, 2026

(54) METHOD FOR DETERMINING A CELL DENSITY OF A DOSING LIQUID, DOSING APPARATUS AND BIOREACTOR

(71) Applicant: Festo SE & Co. KG, Esslingen (DE)

(72) Inventors: Thomas HAHN, Sudbury, MA (US); Sidhaant GUPTA, Waltham, MA (US); Neha MALAVIYA, Worcester, MA (US)

(21) Appl. No.: 19/042,780

(22) Filed: Jan. 31, 2025

(51) Int. Cl. C12M 1/34; C12M 1/00; C12M 1/26

(52) U.S. Cl. CPC C12M 41/36; C12M 23/40; C12M 33/04

(57) ABSTRACT

A method for determining a cell density of a dosing liquid using a dosing apparatus with a fluid channel, a valve, a pressure source, a first dosing liquid reservoir, a processing apparatus, and a fluid channel pressure sensor. The fluid channel includes a first fluid channel section and a second fluid channel section fluidically connected to the first fluid channel section. The valve includes a valve member that is arranged between the first and second fluid channel sections and can be moved into an open position, in which the fluidic connection of the first fluid channel section to the second fluid channel section is opened, and into a closed position, in which the fluidic connection of the first fluid channel section to the second fluid channel section is closed. The first dosing liquid reservoir is fluidically connected to the first fluid channel section, and the dosing liquid includes biological cells.

### BACKGROUND OF THE INVENTION

The invention relates to a method for determining a cell density of a dosing liquid, a dosing apparatus and a bioreactor.

Optical sensors are used in known methods for determining a cell density of a liquid comprising biological cells. The liquid to be examined is applied to a sample carrier. The sample carrier is then examined microscopically using a microscope. If necessary, a contrast agent is also applied to the sample carrier to increase the contrast or improve the visibility of the biological cells. The microscopic examination includes an optical evaluation of an image taken with the microscope, in which biological cells are counted in defined surface areas of the sample or the recorded image. A cell density is then extrapolated based on the counting result. According to the aforementioned measurement method, a corresponding liquid sample is handled outside of a reservoir used for an actual biotechnological method.

### SUMMARY OF THE INVENTION

The object of the present invention is to determine a cell density of a dosing liquid in a simple, fast and reliable manner.

The object is solved by a method described hereinafter, a dosing apparatus described hereinafter, and a bioreactor described hereinafter.

In an method for determining a cell density of a dosing liquid according to the invention, a dosing apparatus is used, which comprises a fluid channel, a valve, a pressure source, a first dosing liquid reservoir in which a dosing liquid is accommodated, a processing apparatus, and a fluid channel pressure sensor associated with the fluid channel, wherein the fluid channel comprises a first fluid channel section and a second fluid channel section fluidically connected to the first fluid channel section, the valve comprising a valve member that is arranged between the first and second fluid channel sections and can be moved into an open position, in which the fluidic connection of the first fluid channel section to the second fluid channel section is opened, and into a closed position, in which the fluidic connection of the first fluid channel section to the second fluid channel section is closed, the first dosing liquid reservoir being fluidically connected to the first fluid channel section, the dosing liquid comprising biological cells.

The method according to the invention comprises the steps of: pressurizing the first dosing liquid reservoir with a pressurized fluid by means of the pressure source so that the dosing liquid is moved by pressurization in the first fluid channel section, moving the valve member from the closed position to the open position at the beginning of a dosing time so that the dosing liquid is moved into the second fluid channel section and flows out of the second fluid channel section, moving the valve member from the open position to the closed position at the end of the dosing time, determining a fluid pressure curve during the dosing time and/or after the dosing time using the fluid channel pressure sensor, wherein the processing apparatus processes the fluid pressure curve in order to determine a cell density of the dosing liquid.

The dosing liquid is moved in the fluid channel. The numbering of the fluid channel sections corresponds to the direction of movement of the dosing liquid within the fluid channel during the dosing process, i.e., starting from the first dosing liquid reservoir. Accordingly, the dosing liquid enters the fluid channel via the first fluid channel section and flows out of the fluid channel via the second fluid channel section.

The movement of the dosing liquid in the fluid channel is initiated by the pressure fluid, which is used to exert pressure on the dosing liquid taken up in the first dosing liquid reservoir. This results in an overpressure in the first dosing liquid reservoir compared to an environment surrounding the dosing apparatus, which is reduced in the direction of the second fluid channel section. In an operating state, the first dosing liquid reservoir comprises at least one first opening through which the first fluid channel section protrudes from the first dosing liquid reservoir. Alternatively or in addition, the first fluid channel section is integrally formed on the first dosing liquid reservoir, so that the first fluid channel section projects from the first dosing liquid reservoir at the first opening.

On the side of the first fluid channel section facing away from the second fluid channel section, the first fluid channel section is immersed in the dosing liquid. This ensures that the dosing liquid flows into the fluid channel when there is overpressure in the dosing liquid reservoir.

The pressure source is used to exert the pressure fluid on the first dosing liquid reservoir. This exerts a corresponding pressure on the first dosing liquid reservoir. A gas, i.e., a compressible fluid, or a liquid, i.e., an incompressible fluid, can be used as the pressure fluid. If a gas is used as the pressure fluid, in particular compressed air, the first dosing liquid reservoir may comprise a second opening through which the pressure fluid in the form of a gas is fed into the first dosing liquid reservoir. A pressure fluid channel connected to the pressure source by means of a fluidic connection is preferably connected to the second opening, in particular integrally formed on the first dosing liquid reservoir.

The first dosing liquid reservoir can be configured so that for a corresponding pressurization, pressurized fluid is introduced into the first dosing liquid reservoir. This is particularly advantageous when a gas is used as the pressure fluid, since in this case, mixing of the pressure fluid with the dosing liquid is not possible due to the principle of operation. Preferably, if pressure fluid is introduced into the first dosing liquid reservoir for a corresponding application, the first dosing liquid reservoir comprises a rigid outer wall.

Alternatively, the first dosing liquid reservoir can be configured in such a way that pressurized fluid is applied to the first dosing liquid reservoir from the outside for a corresponding pressurization. This is particularly advantageous if a liquid is used as the pressurized fluid, since, although mixing of the pressurized fluid with the dosing liquid is possible in principle, it can be prevented by taking the above into account. The first dosing liquid reservoir preferably comprises a flexible outer wall when pressurized fluid is applied from the outside to the first dosing liquid reservoir for a corresponding pressurization.

For the purpose of the present invention, the fluid channel is divided into fluid channel sections in order to be able to clearly identify and assign the different functions or areas of the fluid channel. Preferably, the first and/or second fluid channel section is hose-like and, furthermore, preferably connected to the valve by means of a hose coupling.

When the valve member is in the closed position, the valve member blocks the fluid channel in a fluid-tight manner, preventing the dosing liquid from passing from the first fluid channel section to the second fluid channel section. This also prevents dosing liquid coming from the first dosing liquid reservoir from flowing out of the second fluid channel section. If the valve member is in the open position, the valve member releases the fluid channel so that dosing liquid can pass from the first fluid channel section to the second fluid channel section.

The dosing liquid comprises biological cells. A biological cell is a living unit of an organism. The dosing liquid is used in particular in the field of biotechnology, for example for therapeutic purposes, such as leukemia therapy, the production of tissues, such as artificial organs, but also in brewing or fermentation processes, such as beer brewing using yeast cells. In particular, biological cells that may be considered include mammalian cells, such as blood or stem cells, differentiated cells from tissues, and natural or genetically modified microorganisms that are used in the production of biomolecules and pharmaceuticals or fermentation processes. The dosing liquid is in particular an aqueous, i.e., low-viscosity, in particular water-based or organic, for example alcohol-based, liquid or a corresponding liquid mixture. According to the invention, the cell density of the dosing liquid is determined. The cell density of the dosing liquid corresponds to the number of biological cells in a defined volume of the dosing liquid. The more biological cells present in the defined volume, the higher the cell density of the dosing liquid. The fewer biological cells present in the defined volume, the lower the cell density of the dosing liquid.

Preferably, the fluid channel pressure sensor is configured as a relative pressure sensor. Thus, the fluid pressure curve or fluid pressure is measured directly as the relative pressure with respect to the atmosphere, that is, as a fluid relative pressure. Furthermore, the fluid channel pressure sensor is preferably configured as a piezoresistive pressure sensor. Alternatively, the fluid channel pressure sensor is configured as a piezoelectric or capacitive pressure sensor. The fluid channel pressure sensor is preferably assigned to the first fluid channel section.

If compressed air is used as the pressure fluid, the pressure source is preferably configured as a decentralized compressed air generator, so that the pressure source can be directly assigned to the first dosing liquid reservoir. A pressure source configured in this way comprises a compressor, a power supply and, in particular, a proportional pressure control.

Preferably, a pressure control valve, in particular a proportional pressure control valve, is arranged between the pressure source and the first dosing liquid reservoir, by means of which the pressure exerted on the first dosing liquid reservoir can be controlled. Preferably, the pressure control valve is arranged directly in front of the first dosing liquid reservoir, and particularly preferably in the first dosing liquid reservoir. This is particularly advantageous when a gas, particularly compressed air, is used as the pressure fluid. In this case, the pressure of the compressible gas can be controlled in the immediate vicinity of the point at which the dosing liquid is moved into the fluid channel by means of the pressure fluid. In this way, a fast, i.e., high-frequency, control of the pressure of the pressure fluid can be carried out, with which the dosing liquid is moved into the fluid channel. This enables a controlled movement of the dosing liquid in the fluid channel and a controlled outflow of the dosing liquid from the second fluid channel section. In particular, this can be used to achieve a desired drop break-off of the dosing liquid at the second fluid channel section towards the end of the dosing process.

The processing apparatus is configured to carry out the method according to the invention. For this purpose, the processing apparatus preferably comprises at least one electrical circuit, in particular a microprocessor or microcontroller, on which a computer program code for carrying out the method according to the invention is stored and/or with which the computer program code for carrying out the method according to the invention can be executed. Furthermore, the processing apparatus is preferably set up to detect the fluid pressure curve detected by the fluid channel pressure sensor and to store it, in particular in the processing apparatus.

The method according to the invention is intended, for example, for use in environments with a high degree of automation. By determining the cell density of the dosing liquid using the fluid pressure curve, as provided for in the invention, no further measuring equipment is required to determine the cell density of the dosing liquid, in particular no optical sensors or microscopes.

The dosing time is defined as the time difference or time interval between the time at the beginning of dosing and the time at the end of dosing. The time at the beginning of dosing is the time at which a fluid connection exists between the first fluid channel section and the second fluid channel section after the movement of the valve member from the closed position to the open position has been initiated. For example, when using a valve designed as a diaphragm valve, this is the time at which the diaphragm lifts off a valve seat due to the movement of the valve member. The time at the end of dosing is considered to be the time at which the fluidic connection between the first fluid channel section and the second fluid channel section is interrupted after the movement of the valve member from the open position to the closed position has been initiated. For example, when using a valve configured as a diaphragm valve, this is the time at which the diaphragm again makes a seal with the valve seat.

The fluid pressure curve is a function of time for the fluid pressure detected by the fluid channel pressure sensor. The fluid pressure curve may comprise different fluid pressures at different times, in particular the fluid pressure curve rises up to an extreme point formed as a maximum and falls up to an extreme point formed as a minimum, after which it falls away again or rises.

The inventors have come to the surprising conclusion that the fluid pressure curve is influenced by the viscosity of the dosing liquid and that, if the fluid pressure curve is known, information can be obtained about the viscosity of the dosing liquid. The inventors have also come to the surprising conclusion that the cell density of the dosing liquid influences its viscosity and that as a result, information about the cell density of the dosing liquid can be obtained from the fluid pressure curve.

When it comes to the viscosity of liquids, a distinction is made between dynamic viscosity and kinematic viscosity, although there is a proportional relationship between the two. In particular, the kinematic viscosity of a liquid is the quotient of the dynamic viscosity of the liquid as the dividend and the density of the liquid as the divisor. In other words, the dynamic viscosity of the liquid is the product of the kinematic viscosity of the liquid and the density of the liquid. If the kinematic viscosity of the liquid increases, the dynamic viscosity of the liquid also increases. If the kinematic viscosity of the liquid decreases, the dynamic viscosity of the liquid also decreases.

The physical and biological interactions described below can potentially influence the viscosity of the dosing liquid. There is an interaction between the individual aspects in that, for example, an aspect that increases viscosity can be counterbalanced by another aspect that reduces viscosity, or an aspect that increases viscosity can be increased by another aspect that also increases viscosity.

The biological cells take up space in the dosing liquid and impede the flow of liquid molecules surrounding the biological cells. The more biological cells the dosing liquid comprises, the greater the viscosity of the dosing liquid. The fewer biological cells the dosing liquid comprises, the lower the viscosity of the dosing liquid. Accordingly, assuming a constant average size of biological cells, the viscosity of a dosing liquid with a high cell density will be greater than the viscosity of a dosing liquid with a low cell density.

The biological cells comprise a more or less pronounced deformability or flexibility, depending on the type. When the dosing liquid moves, the biological cells present in the dosing liquid are also moved relative to the dosing liquid, causing the moved biological cells to deform. The deformation can be at least partially elastic. As a result of this deformation, kinetic energy of the dosing liquid is converted into corresponding deformation energy, so that a resistance opposite to the movement of the dosing liquid results. This resistance contributes to the viscosity of the dosing liquid. The viscosity of the dosing liquid is higher the more deformation-related resistance there is, i.e., the higher the deformability of the biological cells. If the biological cells are less deformable, the dosing liquid will be less viscous.

In addition, the viscosity can be influenced by an interaction of the biological cells with each other, for example by adhesion. The denser the biological cells are present, the stronger the interaction between the biological cells and the stronger, for example, a sum of the adhesion forces between the biological cells. This increases the viscosity of the dosing liquid. The lower the interaction between the biological cells, for example because the biological cells are less dense, the lower the viscosity of the dosing liquid. Accordingly, in the case of a dosing liquid with a high cell density, the viscosity is increased even more by the increased interaction of the biological cells with each other compared to a dosing liquid with a low cell density.

Furthermore, the biological cells have a higher density than the dosing liquid without biological cells. Accordingly, a dosing liquid comprising many biological cells has a higher density than a dosing liquid comprising few biological cells. In this respect, too, the viscosity of a dosing liquid is increased by the presence of more biological cells in it.

Taking the above into account, it can be assumed that a dosing liquid with a high cell density comprises a higher viscosity or dynamic viscosity than a dosing liquid with a low cell density.

Preferably, the second fluid channel section comprises a dosing tip, the dosing tip being arranged at an end of the second fluid channel section that faces away from the first fluid channel section, the dosing liquid flowing out of the dosing tip due to the dosing liquid flowing out of the second fluid channel section. The second fluid channel section and the dosing tip can be formed as a single piece. Alternatively, the second fluid channel section and the dosing tip are formed separately and are therefore provided as separate components.

Preferably, the processing apparatus additionally processes a geometric characteristic value of the dosing tip in order to determine the cell density of the dosing liquid. The dosing tip comprises a known internal geometry that is in contact with the dosing liquid. The geometric characteristic value of the dosing tip is determined on the basis of the dimensions of the internal geometry of the dosing tip. Preferably, the internal geometric dimensions of the dosing tip are known within a certain tolerance range, so that the geometric characteristic value of the dosing tip can be determined without a prior separate measurement of the internal geometry of the dosing tip. Preferably, the internal geometry of the dosing tip is rotationally symmetrical, optionally at least partially circular-cylindrical and tapered conically towards a dosing opening. Furthermore, preferably, the radius and/or the length of the cavity of the dosing tip is taken into account when determining the geometric characteristic value of the dosing tip. Especially preferably, the geometric characteristic value of the dosing tip is obtained as the quotient of a divisor and a dividend, wherein the dividend corresponds to the fourth power of the radius of the cavity of the dosing tip and wherein the divisor corresponds to the length of the cavity of the dosing tip.

In principle, all components of the dosing apparatus through which the dosing liquid is moved act as a throttle due to friction and/or changes in cross-section. In addition to the dosing tip, the fluid channel is also important in this regard. However, the dosing tip, which typically tapers to the dosing opening, preferentially exerts a resistance to the movement of the dosing liquid that is at least twice as high as the resistance of the other components of the dosing apparatus. Thus, the dosing tip is intentionally used as a throttle, while the throttle effect of the other components has to be accepted due to the principle involved. The dosing tip's throttling effect can be influenced by its internal geometry. For example, increasing the length of the dosing tip cavity and/or reducing the radius of the dosing tip cavity increases the resistance to the dosing liquid and thus increases the throttling effect. In addition, the resistance offered to the dosing liquid can be reduced, for example by decreasing the length of the dosing tip cavity and/or by increasing the radius of the dosing tip cavity, thus decreasing the throttling effect.

Preferably, a fluid pressure oscillation course is determined by means of the fluid channel pressure sensor immediately after the valve member has been moved from the closed position into the open position or immediately after the valve member has been moved from the open position into the closed position, wherein the cell density of the dosing liquid is determined on the basis of the fluid pressure oscillation course.

The movement of the valve member influences the movement of the dosing liquid in the fluid channel. After moving the valve member from the closed position to the open position, there is an abrupt increase in pressure in the second fluid channel section, which generates a pressure wave in the second fluid channel section. If this pressure wave encounters an obstacle, in particular the dosing tip acting as a throttle, the pressure wave is at least partially reflected at the obstacle, resulting in a pressure oscillation. The temporal course of this pressure oscillation is referred to as the fluid pressure oscillation course with respect to the fluid pressure. The fluid pressure oscillation course is determined by the fluid pressure sensor and is preferably a time-related part of the fluid pressure curve, which is in particular directly after the valve member has moved from the closed position into the open position, i.e. at the start of the dosing time, or directly after the valve member has moved from the open position into the closed position, i.e. at the end of the dosing time.

In the same way, a pressure oscillation occurs when the valve member is moved from the open position to the closed position. In this case, there is an abrupt pressure drop. In the area of the second fluid channel section facing the first fluid channel section, the pressure is then lower than in the rest of the second fluid channel section, at least briefly. This pressure difference leads to a pressure equalization that is directed against the direction of movement of the dosing liquid during dosing.

Accordingly, the fluid pressure oscillation course can be determined both after moving the valve member from the closed position to the open position and after moving the valve member from the open position to the closed position.

The fluid pressure oscillation course depends on the dynamic and/or kinematic viscosity of the dosing liquid. The kinematic viscosity is a measure of how the dosing liquid flows under the influence of gravity. The dynamic viscosity is a measure of the resistance of the dosing liquid to deformation. Since the pressure oscillation curve, as described above, results from an influence on the movement of the dosing liquid, a dosing liquid with a high dynamic viscosity exerts greater resistance to the movement influence than a dosing liquid with a low dynamic viscosity. Thus, the pressure curve or fluid pressure oscillation course of a dosing liquid with a high dynamic viscosity and thus high cell density is less affected than the pressure curve or fluid pressure oscillation course of a dosing liquid with a low dynamic viscosity and thus low cell density.

On the one hand, this can be expressed in the fact that lower pressure amplitudes are achieved with a dosing liquid with a high dynamic viscosity than with a dosing liquid with a low dynamic viscosity. This is due to the fact that with a dosing liquid with a high dynamic viscosity, there is a stronger damping of the pressure oscillation associated with the pressure amplitudes. This type of damping is also referred to as viscous damping. On the other hand, this can be manifested by the fact that the frequency of the pressure amplitudes is greater for a dosing liquid with a high dynamic viscosity than for a dosing liquid with a low dynamic viscosity. Furthermore, this can be expressed in the fact that for a dosing liquid with a high dynamic viscosity, the decay rate of the amplitudes of successive pressure extreme values is greater, i.e., the pressure oscillation is damped more than for a dosing liquid with a low dynamic viscosity.

Taking into account the relationships described above between the fluid pressure oscillation course and the dynamic and/or kinematic viscosity of the dosing liquid, as well as the viscosity of the dosing liquid and the biological and/or physical influences of the biological cells of the dosing liquid, it is possible to draw conclusions about the cell density of the dosing liquid from the fluid pressure oscillation course or from the characteristic values derived characteristic values derived from the fluid pressure oscillation curve on the cell density of the dosing liquid.

Preferably, the cell density of the dosing liquid is determined with at least one characteristic value of the group: amplitude of the first extremum, amplitude of the second extremum, amplitude of the third extremum, time difference between the first extremum and/or the second extremum and/or the third extremum, decay rate of the amplitude of at least two successive extremums, which characteristic value is determined with the aid of the fluid pressure vibration curve. Taking into account the mechanisms described above, conclusions can be drawn from the aforementioned amplitude-related characteristic values regarding the viscosity of the dosing liquid and thus the cell density of the dosing liquid.

Amplitude is defined as the difference between the relevant extremum and a normal pressure, in particular the difference between the relevant extremum and a fluid pressure that occurs when closing or opening is constant.

The numbering of the amplitude-related extremums follows the temporal occurrence of the respective extremums, i.e., the first extremum occurs first, followed by the second extremum. Then the third extremum occurs. It is possible to use the amplitudes of further extremums as characteristic values for determining the cell density of the dosing liquid, for example the amplitude of the fourth, fifth, sixth, seventh, eighth, ninth and/or tenth extremum. The extremums can be maximum or minimum in each case.

The time intervals between the extremums may include, but are not limited to, the time between the first and second extremums, the time between the first and third extremums, and the time between the second and third extremums. If further extremums are determined or taken into account, further time intervals can be considered iteratively, for example the time between the first and the fourth extremum, the time between the second and the fourth extremum and the time between the third and the fourth extremum, and so on.

The decay rate of the amplitude of at least two successive extremums is a measure of how quickly the amplitude values of successive extremums decrease. With a high decay rate, the amplitude values decrease more strongly or more quickly than with a low decay rate.

Preferably, before determining the cell density of the dosing liquid, the processing apparatus is parameterized by carrying out the following steps for at least one test liquid of known cell density: moving the test liquid in the fluid channel by means of moving the valve member from the closed position to the open position and then moving the valve member from the open position to the closed position, wherein in connection with the movement of the test liquid in the fluid channel immediately after the valve member has been moved from the closed position to the open position or immediately after the valve member has been moved from the open position to the closed position, a test fluid pressure oscillation course is determined with the fluid channel pressure sensor, the cell concentration of the test liquid being determined on the basis of the test fluid pressure oscillation course, the cell concentration of the test liquid being determined with at least one characteristic value of the group: amplitude of the first extremum, amplitude of the second extremum, amplitude of the third extremum, time difference between the first extremum and/or the second extremum and/or the third extremum, decay rate of the amplitude of at least two successive extremums, which characteristic value is determined with reference to the test fluid pressure oscillation course, at least one cell density class being then determined taking into account the determined cell density of the at least one test liquid, the at least one cell density class being assigned in the subsequent determination of the cell density in the event of corresponding dosing liquid, the at least one cell density class is assigned, wherein the cell density of the dosing liquid is determined with this assigned at least one cell density class.

By determining the cell concentration of the at least one test liquid and, based on this, determining a cell density class, the direct determination of the cell density of the dosing liquid, for example on the basis of the fluid pressure oscillation course, can be dispensed with. Rather, the associated cell density class required for determining the cell density of the dosing liquid is used in accordance with the preferred further development of the invention as set out above. This associated cell density class, once determined and stored in the processing apparatus, for example, can be used in place of the cell density of the respective dosing liquid for subsequent dosing processes. If reference is made to the cell density of the dosing liquid, this may refer to an assigned cell density class, but it may also refer to a cell density of the dosing liquid that has been determined, for example, on the basis of the fluid pressure oscillation course without prior knowledge of at least one cell density class.

Preferably, the assignment of a cell density class to the respective dosing liquid is less complex than the determination of a respective cell density of the dosing liquid without prior knowledge of at least one cell density class. In particular, the assignment to a cell density class is carried out on the basis of a few amplitude-related characteristic values, or possibly on the basis of only one amplitude-related characteristic value, while the determination of the cell density of the test liquid in question is carried out on the basis of more amplitude-related characteristic values.

If the fluid pressure oscillation course is determined after the valve member has been moved from the open position to the closed position, the time between the movement of the valve member from the closed position to the open position and the movement of the valve member from the open position to the closed position can be minimized. Alternatively, or in addition, a test dosing time can be used as the time between the movement of the valve member from the closed position to the open position and the movement of the valve member from the open position to the closed position.

Preferably, a maximum feed pressure provided by the pressure source is also taken into account in the parameterization of the processing apparatus, which is determined by the fluid channel pressure sensor while the valve member is in the closed position. It is particularly preferred that several different maximum feed pressures be taken into account. This can increase the accuracy of determining the cell density of the dosing liquid, even when using different maximum feed pressures.

Preferably, the processing apparatus is parameterized with a plurality of test liquids having different known cell densities, wherein a plurality of cell density classes is determined taking into account the cell density determined for each of the plurality of test liquids. This can increase the accuracy of the determination of the cell concentration of the dosing liquid. The cell density class can also be regarded as the corresponding cell density with a certain standard deviation. Furthermore, a cell density is preferably determined several times for a test liquid, and the corresponding cell density class is determined on the basis of the several cell densities of a test liquid.

The known different cell densities used for the parameterization of the processing apparatus are preferably determined by means of previously known measuring methods, for example by means of the aforementioned microscope-based counting method.

Preferably, the different viscosity classes are determined using a cell concentration-based model for machine learning. The cell-concentration-related model for machine learning is a model in which at least one statistical learning algorithm is stored and with which functional relationships can be determined between at least one cell concentration of the dosing liquid as an input variable and, if applicable, other input variables and at least one cell density class as an output variable. This makes it possible to avoid the complex task of acquiring knowledge of physical relationships concerning the dosing liquid and the dosing apparatus, which in particular simplifies the parameterization of the processing apparatus.

The cell-concentration-based model is preferably trained for machine learning after at least one cell density class has been determined. In particular, after the cell-concentration-based model has been trained for machine learning, the cell density of the dosing liquid is determined taking into account the trained cell-concentration-based model for machine learning.

Preferably, the dosing apparatus comprises a second dosing liquid reservoir, the second dosing liquid reservoir being fluidically connected to the second fluid channel section, the dosing liquid flowing out of the second fluid channel section flowing into the second dosing liquid reservoir. As a result, the dosing liquid flowing out of the second fluid channel section can be reused immediately, in particular, for biotechnological purposes. Such biotechnological reuse may, for example, involve the further cultivation of the said dosing liquid or the extraction of biological cells from the said dosing liquid.

Preferably, a difference of at least 30 mbar, more preferably of at least 45 mbar, especially of at least 60 mbar is present between a first internal pressure formed in the first dosing liquid reservoir and a second internal pressure formed in the second dosing liquid reservoir. Furthermore, the first internal pressure is preferably higher than the second internal pressure. On the one hand, this can better ensure that dosing liquid is moved from the first dosing liquid reservoir to the second dosing liquid reservoir. On the other hand, this can better ensure that the fluid pressure oscillation course occurs to such an extent that the fluid pressure oscillation course can be easily determined.

Preferably, the dosing apparatus is part of a bioreactor, wherein the dosing liquid further comprises a nutrient. Furthermore, the dosing liquid may comprise growth factors and/or a buffer solution. The buffer solution can serve to buffer, for example, the pH of the dosing liquid. Furthermore, both dosing liquid reservoirs are preferably part of the bioreactor. Most preferably, the dosing apparatus is part of the bioreactor. The bioreactor is an apparatus used to cultivate biological cells and to create a biologically active environment. This makes it easy to determine the progress of cell cultivation, especially in-line. Nutrient refers to any substance that biological cells can metabolize. For example, glucose can be used as a nutrient. The bioreactor is preferably configured to influence the activity, or the growth of the biological cells accommodated in the relevant reservoir of dosing liquid. For this purpose, the bioreactor can comprise, for example, a corresponding temperature control, a nutrient supply, a process gas supply, in particular an oxygen or air supply, and/or a stirrer.

Preferably, the dosing apparatus further comprises a nutrient sensor, wherein a nutrient concentration of the dosing liquid is determined with the nutrient sensor, wherein the processing apparatus additionally processes the nutrient concentration of the dosing liquid to determine the cell density of the dosing liquid. The nutrient concentration corresponds to the amount of nutrient in a defined volume of the dosing liquid. The nutrient(s) present in the dosing liquid and thus the nutrient concentration can influence the viscosity of the dosing liquid and thus, taking into account the above interrelationships, the fluid pressure curve. If the nutrient concentration is not known, a certain inaccuracy results when determining the cell density. To reduce the influence of the nutrient concentration and thus the inaccuracy associated with it when determining the cell density, it is advantageous to take into account the nutrient concentration of the dosing liquid as described above.

The nutrient sensor can be assigned to the fluid channel and/or at least one of the dosing liquid reservoirs, for example the first dosing liquid reservoir and/or the second dosing liquid reservoir. The nutrient sensor may be configured as a glucose sensor. The dosing apparatus may further comprise an oxygen sensor, a carbon dioxide sensor, an electrolyte sensor (for example an ion-selective sensor) and/or another sensor with which extracellular components can be measured.

Alternatively or according to a preferred embodiment, the fluid channel pressure sensor is arranged in such a way that the fluid channel pressure sensor does not directly contact the sample liquid, wherein the pressure source is configured as a hydraulic pressure source and a hydraulic fluid is used as pressure fluid, wherein the first dosing liquid reservoir is pressurized from the outside with the pressure fluid by means of the pressure source. A pressurization of the first dosing liquid reservoir from outside means that the pressurized fluid does not come into contact with the dosing liquid.

Preferably, the dosing apparatus further comprises a first reservoir pressure sensor associated with the first dosing liquid reservoir, wherein during the determination of the fluid pressure profile with the first reservoir pressure sensor, a first internal pressure formed in the first dosing liquid reservoir is determined, wherein the processing apparatus additionally processes the first internal pressure to determine the cell density of the dosing liquid. This can reduce the influence of the level of the dosing liquid accommodated in the first dosing liquid reservoir on the determination of the cell density of the dosing liquid. A higher level in the first dosing liquid reservoir leads to a higher first internal pressure, independently of the admission of pressurized fluid to the first dosing liquid reservoir, which in turn results in a higher fluid pressure. This influence leads to a certain inaccuracy in determining the cell density of the dosing liquid, but this can be reduced by taking into account the first internal pressure. Similarly, or in addition, a first level sensor associated with the first dosing liquid reservoir can be provided.

Preferably, the dosing apparatus further comprises a second reservoir pressure sensor associated with the second dosing liquid reservoir. Furthermore, during the determination of the fluid pressure curve with the second reservoir pressure sensor, a second internal pressure present in the second dosing liquid reservoir is determined, with the processing apparatus additionally processing the first internal pressure in order to determine the cell density of the dosing liquid. Alternatively or in addition, a second level sensor is provided that is assigned to the second dosing liquid reservoir.

A dosing apparatus according to the invention comprises a fluid channel, a valve, a pressure source, a first dosing liquid reservoir in which a dosing liquid can be accommodated, a processing apparatus, and a fluid channel pressure sensor associated with the fluid channel, wherein the fluid channel comprises a first fluid channel section and a second fluid channel section fluidically connected to the first fluid channel section, wherein the valve comprises a valve member that is arranged between the first and second fluid channel sections and can be moved into an open position, in which the fluidic connection of the first fluid channel section to the second fluid channel section is opened, and into a closed position, in which the fluidic connection of the first fluid channel section to the second fluid channel section is closed, wherein the first dosing liquid reservoir is fluidically connected to the first fluid channel section, wherein the dosing liquid comprises biological cells, wherein the processing apparatus is configured to carry out a method comprising the steps of: pressurizing the first dosing liquid reservoir with a pressurized fluid by means of the pressure source so that the dosing liquid is moved by pressurization in the first fluid channel section, moving the valve member from the closed position to the open position at the beginning of a dosing time so that the dosing liquid is moved into the second fluid channel section and flows out of the second fluid channel section, moving the valve member from the open position to the closed position at the end of the dosing time, determining a fluid pressure curve during the dosing time and/or after the dosing time using the fluid channel pressure sensor, wherein the processing apparatus processes the fluid pressure curve in order to determine a cell density of the dosing liquid. The above considerations regarding the method according to the invention in connection with the individual components of the dosing apparatus apply equally to the components of the dosing apparatus according to the invention.

Preferably, the valve is configured as a pinch valve. The pinch valve encompasses the fluid channel, which, in particular, is configured as a flexible hose, at least in the relevant area. To move the valve member into the closed position, the valve member is pressed from the outside onto the fluid channel in such a way that an inside diameter of the fluid channel is reduced locally to such an extent that the transport of dosing liquid is prevented in the relevant area. The fluid channel is accordingly pinched locally. This can prevent the valve, in particular the valve member, from coming into contact with the dosing liquid. In this way, the method according to the invention can be carried out in a sterile manner, which is advantageous, for example, when using dosing liquids in the production of cell cultures for therapeutic purposes (CAR-T cell production) or other preparative cell culture methods. Furthermore, the pressure source is preferably configured as a peristaltic pump.

Alternatively, or in addition, the valve can be configured as a media-separated diaphragm valve. The valve can also be configured as a piston slide valve or a seat valve.

Preferably, the valve is configured to be solenoid-operated. This means that the valve is configured as a solenoid valve. The movement of the valve member is effected by energizing an electromagnet. The movement of the valve member can optionally release or block a valve seat formed between the first fluid channel section and the second fluid channel section.

Preferably, the dosing apparatus comprises a temperature sensor that can be used to determine the temperature of the dosing liquid. Furthermore, when determining the cell density of the dosing liquid, the temperature of the dosing liquid is taken into account. The activity or movement or mobility of some biological cells is influenced by their temperature or the temperature of the dosing liquid in which the biological cells are accommodated. In a certain temperature range, the activity of the biological cells increases with temperature. Thus, at a high temperature, there is a high activity of the biological cells and at a low temperature, there is a low activity of the biological cells. The activity of the biological cells, in turn, can influence the viscosity of the dosing liquid and thus, taking into account the aforementioned cause-and-effect relationships, the fluid pressure curve. This may result in a certain inaccuracy when determining the cell density of the dosing liquid, which in turn can be reduced by taking into account the temperature of the dosing liquid when determining the cell density of the dosing liquid.

A bioreactor according to the invention comprises a dosing apparatus as described above. The above considerations regarding the method according to the invention as applied to the bioreactor also apply to the bioreactor according to the invention.

### BRIEF DESCRIPTION OF THE DRAWINGS

The invention is explained in more detail below, with reference to the accompanying drawings, in which it is shown:

FIG. 1 a dosing apparatus with a valve in which a valve member is in an open position,

FIG. 2 the dosing apparatus shown in FIG. 1, with the valve member in the closed position,

FIG. 3 another dosing apparatus with a dosing tip,

FIG. 4 another dosing apparatus,

FIG. 5 a detailed view of the dosing tip shown in FIG. 3,

FIG. 6 a method for determining the cell density of a dosing liquid,

FIG. 7 a fluid pressure curve of a weakly influenced input pressure,

FIG. 8 a fluid pressure curve of a strongly influenced input pressure,

FIG. 9 a cell density curve,

FIG. 10 a dosing liquid with a high cell density, and

FIG. 11 a dosing liquid with a low cell density.

### DETAILED DESCRIPTION

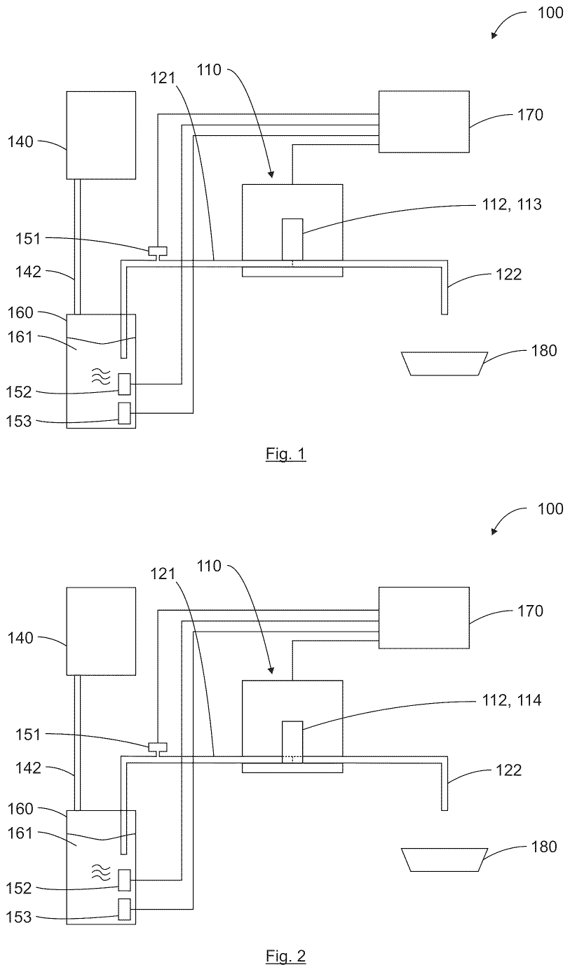

FIG. 1 shows a dosing apparatus 100. The dosing apparatus 100 comprises a valve 110 having a valve member 112. The dosing apparatus 100 comprises a fluid channel that comprises a first fluid channel section 121 and a second fluid channel section 122. The first fluid channel section 121 is fluidically connected to the second fluid channel section 122.

The dosing apparatus 100 further comprises a valve 110 having a valve member 112. The valve member 112 is arranged between the first fluid channel section 121 and the second fluid channel section 122. The valve member 112 can be moved to an open position 113, in which a fluidic connection between the first fluid channel section 121 and the second fluid channel section 122 is enabled. As shown in FIG. 1, with the dosing apparatus 100, the valve member 112 is in the open position 113. The valve member 112 can also be moved from the open position 113 to a closed position 114, in which the fluidic connection of the first fluid channel section 121 with the second fluid channel section 122 is closed. In the dosing apparatus 100 shown in FIG. 2, the valve member 112 is in the closed position 114.

The dosing apparatus 100 comprises a first dosing liquid reservoir 160 in which a dosing liquid 161 is accommodated. The dosing liquid 161 comprises biological cells 163 (see FIGS. 10 and 11). By way of example, the first dosing liquid reservoir 160 is fluidically connected to the first fluid channel section 121 by immersing the first fluid channel section 121 in the dosing liquid 161.

The dosing apparatus 100 comprises a pressure source 140, by means of which the first dosing liquid reservoir 160 can be supplied with a pressurized fluid. For this purpose, the pressure source 140 is connected to the first dosing liquid reservoir 160 by means of a pressure fluid channel 142.

The dosing apparatus 100 comprises a fluid channel pressure sensor 151 associated with the fluid channel. By way of example only, the fluid channel pressure sensor 151 is associated with the first fluid channel section 121, such that the fluid channel pressure sensor 151 is arranged upstream of the valve 110, starting from the first dosing liquid reservoir 160.

By way of example only, the dosing apparatus 100 comprises a nutrient sensor 152, by means of which a nutrient concentration of the dosing liquid 161 can be determined. The nutrient sensor 152 is preferably arranged in the first dosing liquid reservoir 160. It is also preferred that the nutrient sensor 152 is immersed in the dosing liquid 161.

Furthermore, by way of example, the dosing apparatus 100 comprises a first reservoir pressure sensor 153 associated with the first dosing liquid reservoir 160, by means of which a first internal pressure configured in the first dosing liquid reservoir 160 can be determined. The first reservoir pressure sensor 153 is preferably arranged in the first dosing liquid reservoir 160. Furthermore, the first reservoir pressure sensor 153 is immersed in the dosing liquid 161.

The dosing apparatus 100 comprises a processing apparatus 170 that is electrically connected to the fluid channel pressure sensor 151, the nutrient sensor 152, the first reservoir pressure sensor 153 and the valve 110.

As an example, a sample container 180 is arranged behind or under the end of the second fluid channel section 122 facing away from the first fluid channel section 121, in which the dosing liquid 161 flowing out of the second fluid channel section 122 can be accommodated.

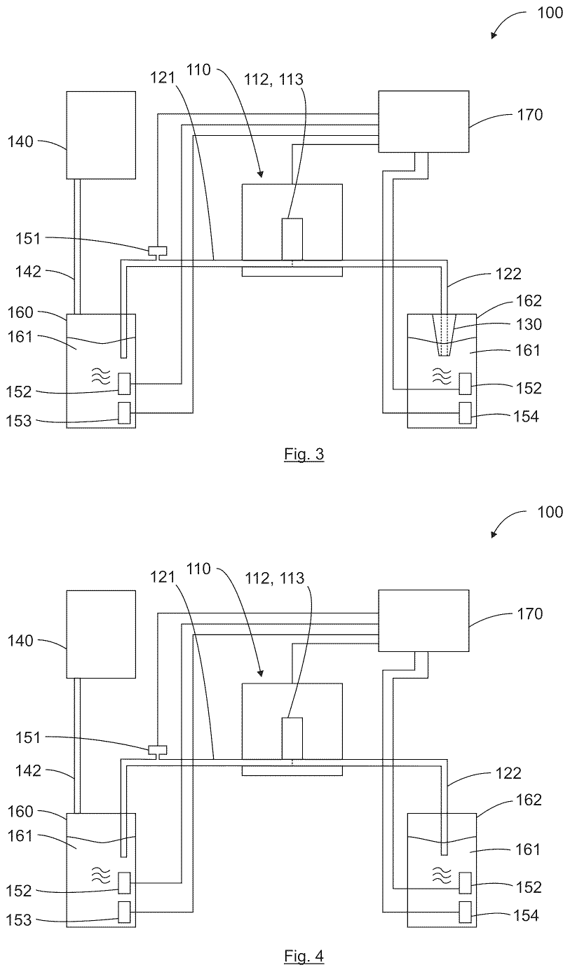

FIGS. 3 and 4 each show a further dosing apparatus 100. The respective dosing apparatus 100 shown in FIGS. 3 and 4 each comprises a second dosing liquid reservoir 162. The second fluid channel section 122 opens into the second dosing liquid reservoir 162.

By way of example, the dosing apparatus 100 comprises a further nutrient sensor 152, which is arranged in the second dosing liquid reservoir 162. The further nutrient sensor 152 can also be used to determine a nutrient concentration of the dosing liquid 161. Like the nutrient sensor 152 associated with the first dosing liquid reservoir 160, the further nutrient sensor 152 associated with the second dosing liquid reservoir 162 is preferably immersed in the dosing liquid 161.

Furthermore, by way of example, the dosing apparatus 100 comprises a second reservoir pressure sensor 154 associated with the second dosing liquid reservoir 162, by means of which a second internal pressure present in the second dosing liquid reservoir 162 can be determined. Preferably, the second reservoir pressure sensor 154 is arranged in the second dosing liquid reservoir 162. It is further preferred that the second reservoir pressure sensor 154 is immersed in the dosing liquid 161.

In the dosing apparatus 100 shown in FIG. 3, the second fluid channel section 122 comprises a dosing tip 130. The dosing tip 130 is arranged at the end of the second fluid channel section 122 facing away from the first fluid channel section 121. The dosing tip 130 acts as a throttle. Exemplarily, the dosing tip 130 is immersed in the dosing liquid 161 accommodated in the second dosing liquid reservoir 162.

With the dosing apparatus 100 shown in FIG. 4, the end of the second fluid channel section 122 facing away from the first fluid channel section 121 is immersed in the dosing liquid 161 accommodated in the second dosing liquid reservoir 162.

FIG. 5 shows the dosing tip 130 of the dosing apparatus 100 shown in FIG. 3. The dosing tip 130 comprises an internal geometry 131 that is in contact with the dosing liquid 161. By way of example only, the internal geometry 131 is configured as a circular cylinder and comprises a length 134 and a radius 132. For example, the outer geometry of the dosing tip 130 is conical. In addition, other outer geometries of the dosing tip 130 are possible, for example, the outer geometry of the dosing tip 130 can be configured as a circular cylinder.

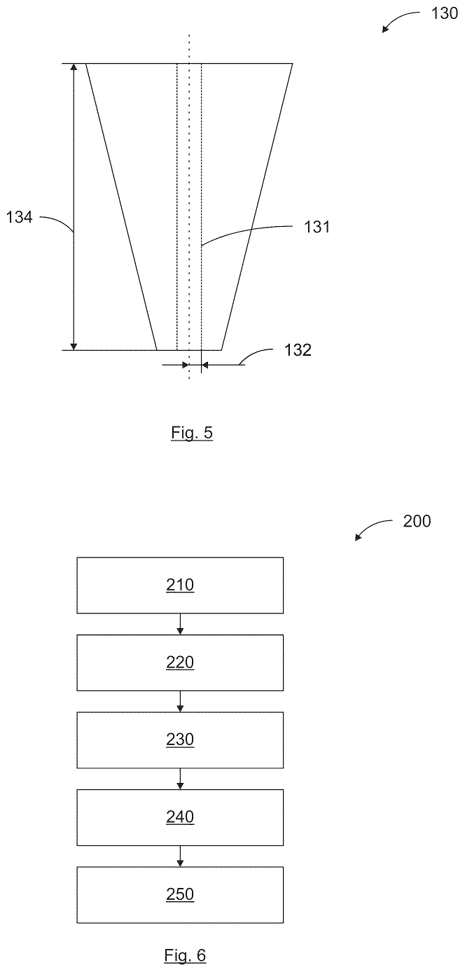

The processing apparatus 170 is configured to carry out the method 200 shown in FIG. 6. The method 200 is carried out using the dosing apparatus 100 described above. In particular, it is assumed here that the fluid channel has been vented beforehand so that the entire fluid channel, i.e., the first fluid channel section 121 and the second fluid channel section 122, is filled with the dosing liquid 161.

The method 200 begins with a first step 210, in which the first dosing liquid reservoir 160 is pressurized by means of the pressure source 140, so that the dosing liquid 161 is moved by pressurization in the first fluid channel section 121.

A second step 220 follows, in which the valve member 112 is moved from the closed position 114 to the open position 113 at the beginning of a dosing time, so that the dosing liquid 161 is moved into the second fluid channel section 122 and flows out of the second fluid channel section 122.

In a subsequent third step 230, the valve member 112 is moved from the open position 113 to the closed position 114 at the end of the dosing time.

A fourth step 240 then follows, in which a fluid pressure curve is determined during the dosing time and/or after the dosing time using the fluid channel pressure sensor 151.

Finally, a fifth step 250 follows in which the processing apparatus 170 processes the fluid pressure curve to determine a cell density of the dosing liquid 161.

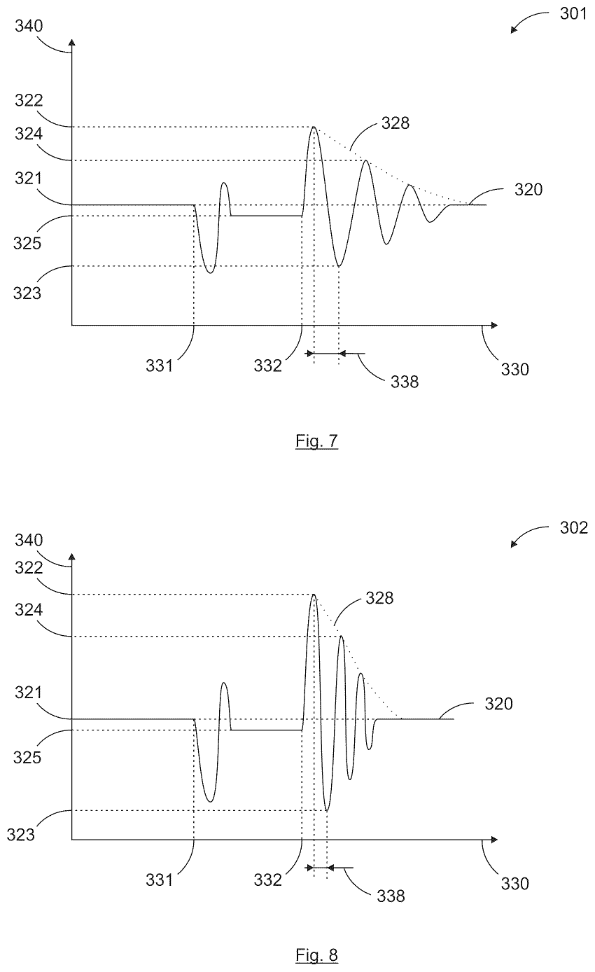

FIG. 7 shows a diagram 301 with a fluid pressure curve 320 of a weakly influenced fluid pressure and FIG. 8 shows a diagram 302 with a fluid pressure curve 320 of a strongly influenced fluid pressure. Assuming otherwise identical conditions, in particular an identical dosing apparatus 100 and an identical pressure provided by the pressure source 140, the fluid pressure curve 320 shown in FIG. 7 corresponds to a dosing liquid 161 that comprises a higher cell density than the dosing liquid 161 to which the fluid pressure In other words, the fluid pressure curve shown in FIG. 7 with the fluid pressure curve 320 is damped more than the fluid pressure curve shown in FIG. 8 with the fluid pressure curve 320.

The fluid pressure curve 320 corresponds to a fluid pressure over time plotted on a pressure axis 340 over a time axis 330. The fluid pressure is determined by the fluid channel pressure sensor 151 (see FIGS. 1 to 4). At the beginning, the fluid pressure corresponds to a first fluid pressure 321. At a time at the beginning of dosing 331, a pressure oscillation results, which manifests itself in the fluid pressure dropping abruptly and then settling at a fifth fluid pressure 325. The fifth fluid pressure 325 is slightly below the first fluid pressure 321. The difference between the first fluid pressure 321 and the fifth fluid pressure 325 is due to the fact that in the open position 113, i.e. between the time at the beginning of dosing 331 and a time at the end of dosing 332, a pressure loss occurs via the fluid channel and in particular via the dosing tip 130.

After the time at the end of dosing 332, a pressure oscillation results, which manifests itself in the fact that the fluid pressure rises abruptly until a maximum is reached as an extremum with a second fluid pressure 322. The fluid pressure then drops until a minimum is reached as an extremum with a third fluid pressure 323. The fluid pressure then rises again until a maximum is reached as an extremum with a fourth fluid pressure 324. Finally, the fluid pressure levels off again at the first fluid pressure 321, whereby a few further extremums are reached, each configured as a maximum or minimum, which are not provided with reference signs in FIGS. 7 and 8 for the sake of clarity.

FIGS. 7 and 8 also each illustrate a time difference 338 between the time at which the fluid pressure reaches the second fluid pressure 322 and the time at which the fluid pressure reaches the third fluid pressure 323. This time difference 338 may depend on the dynamic viscosity of the dosing liquid 161 and thus on its cell density. By way of example only, the time difference 338 shown in FIG. 7, which is associated with a dosing liquid 161 with a high cell density, is greater than the time difference 338 shown in FIG. 8, which is associated with a dosing liquid 161 with a low cell density. The dosing time corresponds to the difference between the time at the end of dosing 332 and the time at the beginning of dosing 331. In addition to the pressure extremums 322, 323, 324, the time difference 338 can be used as a further indicator of the dynamic viscosity of the dosing liquid 161. Nevertheless, it is possible that a difference between the respective pressure extremums 322, 323, 324 can be determined for dosing liquids 161 with different dynamic viscosities, as shown in FIGS. 7 and 8, but no difference in the time difference 338, i.e. in contrast to the fluid pressure curves 320 shown in FIGS. 7 and 8.

The deviations of the amplitude of the input pressure between the pressure extremums 322, 323, 324 and the first fluid pressure 321 is smaller for the fluid pressure curve 320 shown in FIG. 7, which is associated with a dosing liquid 161 with a low cell density, than for the fluid pressure curve 320 shown in FIG. 8, which is associated with a dosing liquid 161 with a high cell density. In addition, a decay rate 328 of the amplitudes of successive pressure extremums 322, 324 for the fluid pressure curve 320 shown in FIG. 8 is greater than the decay rate 328 of the fluid pressure curve 320 shown in FIG. 7.

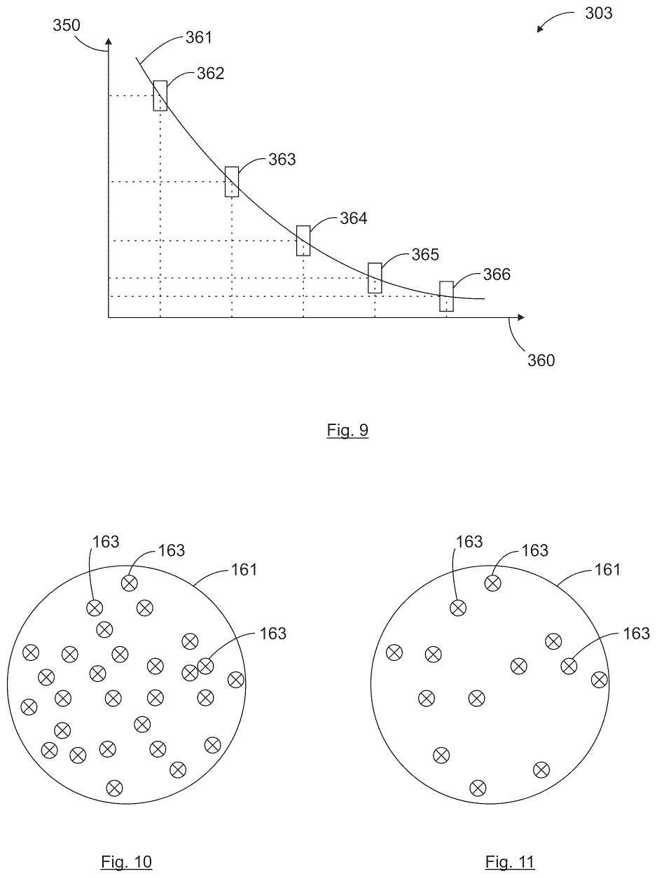

FIG. 9 shows a diagram 303 with a cell density curve 361. The cell density curve 361 corresponds to a curve of a cell density of a dosing liquid 161 plotted on an extremum axis 350 over a cell density axis 360 as a function of an extremum, which is determined on the basis of the fluid pressure curve associated with the dosing liquid 161 (cf. FIGS. 7 and 8). In particular, a difference between the third fluid pressure 323 and the first fluid pressure 321 is used as the extremum. This difference is also referred to as the amplitude of the third extremum. The first fluid pressure 321 functions as the normal pressure, namely as the fluid pressure that is present when the valve 110 is constantly closed. With increasing cell density, i.e., along the cell density axis 360 with increasing values, the extremum plotted on the extremum axis 350 decreases. As an example, the difference between the third fluid pressure 323 and the first fluid pressure 321 decreases with increasing cell density. This can also be understood from the fluid pressure curves 320 shown in FIGS. 7 and 8.

By way of example only, five cell density classes 362, 363, 364, 365, 366 are shown in the diagram 303 shown in FIG. 9, namely a first cell density class 362, a second cell density class 363, a third cell density class 364, a fourth cell density class 365 and a fifth cell density class 366. Each cell density class 362, 363, 364, 365, 366 comprises a certain extremum range, which is expressed by the extension of the respective cell density class 362, 363, 364, 365, 366 along the extremum axis 350, and a certain cell density range, which is expressed by the extension of the respective cell density class 362, 363, 364, 365, 366 along the cell density axis 360. Preferably, neighboring cell density classes 362, 363, 364, 365, 366 are spaced apart with respect to at least one of the extremum axis 350 and cell density axis 360 in such a way that an unambiguous assignment to one of the cell density classes 362, 363, 364, 365, 366 is made possible. In this context, unambiguous assignment means that it is prevented that a pair of values consisting of an extremum and a cell density can be assigned to several cell density classes 362, 363, 364, 365, 366, i.e. that several cell density classes 362, 363, 364, 365, 366 overlap at the point in question.

FIG. 10 shows a dosing liquid 161 with a high cell density and FIG. 11 shows a dosing liquid 161 with a low cell density. As mentioned above, the dosing liquid 161 comprises biological cells 163. In FIGS. 10 and 11, for reasons of clarity, only three biological cells 163 are provided with a reference sign in each case. The dosing liquid 161 shown in FIG. 10 comprises more biological cells 163 than the dosing liquid shown in FIG. 11, which is associated with the fact that the dosing liquid 161 shown in FIG. 10 comprises a higher cell density than the dosing liquid 161 shown in FIG. 11. For example, the dosing liquid 161 shown in FIG. 10 may be associated with the fluid pressure curve 320 shown in FIG. 7 and the dosing liquid 161 shown in FIG. 11 may be associated with the fluid pressure curve 320 shown in FIG. 8.

What is claimed is:

1. A method for determining a cell density of a dosing liquid using a dosing apparatus with a fluid channel, a valve, a pressure source, a first dosing liquid reservoir in which a dosing liquid is accommodated, a processing apparatus, and a fluid channel pressure sensor associated with the fluid channel, wherein the fluid channel comprises a first fluid channel section and a second fluid channel section fluidically connected to the first fluid channel section, the valve comprising a valve member that is arranged between the first and second fluid channel sections and can be moved into an open position, in which the fluidic connection of the first fluid channel section to the second fluid channel section is opened, and into a closed position, in which the fluidic connection of the first fluid channel section to the second fluid channel section is closed, the first dosing liquid reservoir being fluidically connected to the first fluid channel section, the dosing liquid comprising biological cells, the method comprising the steps of:
pressurizing the first dosing liquid reservoir with a pressurized fluid by means of the pressure source so that the dosing liquid is moved by pressurization in the first fluid channel section,
moving the valve member from the closed position to the open position at the beginning of a dosing time so that the dosing liquid is moved into the second fluid channel section and flows out of the second fluid channel section,
moving the valve member from the open position to the closed position at the end of the dosing time,
determining a fluid pressure curve during the dosing time and/or after the dosing time using the fluid channel pressure sensor,
wherein the processing apparatus processes the fluid pressure curve in order to determine a cell density of the dosing liquid.

2. The method according to claim 1, wherein the second fluid channel section comprises a dosing tip, the dosing tip being arranged at an end of the second fluid channel section that faces away from the first fluid channel section, the dosing liquid flowing out of the dosing tip due to the dosing liquid flowing out of the second fluid channel section.

3. The method according to claim 2, wherein the processing apparatus additionally processes a geometric characteristic value of the dosing tip in order to determine the cell density of the dosing liquid.

4. The method according to claim 1, wherein a fluid pressure oscillation course is determined by means of the fluid channel pressure sensor immediately after the valve member has been moved from the closed position into the open position or immediately after the valve member has been moved from the open position into the closed position, wherein the cell density of the dosing liquid is determined on the basis of the fluid pressure oscillation course.

5. The method according to claim 4, wherein the cell density of the dosing liquid is determined with at least one characteristic value of the group: amplitude of the first extremum, amplitude of the second extremum, amplitude of the third extremum, time difference between the first extremum and/or the second extremum and/or the third extremum, decay rate of the amplitude of at least two successive extremums, which characteristic value is determined with the aid of the fluid pressure vibration curve.

6. The method according claim 1, wherein, before determining the cell density of the dosing liquid, the processing apparatus is parameterized by carrying out the following steps for at least one test liquid of known cell density: moving the test liquid in the fluid channel by means of moving the valve member from the closed position to the open position and then moving the valve member from the open position to the closed position, wherein in connection with the movement of the test liquid in the fluid channel immediately after the valve member has been moved from the closed position to the open position or immediately after the valve member has been moved from the open position to the closed position, a test fluid pressure oscillation course is determined with the fluid channel pressure sensor, the cell concentration of the test liquid being determined on the basis of the test fluid pressure oscillation course, the cell concentration of the test liquid being determined with at least one characteristic value of the group: amplitude of the first extremum, amplitude of the second extremum, amplitude of the third extremum, time difference between the first extremum and/or the second extremum and/or the third extremum, decay rate of the amplitude of at least two successive extremums, which characteristic value is determined with reference to the test fluid pressure oscillation course, at least one cell density class being then determined taking into account the determined cell density of the at least one test liquid, the at least one cell density class being assigned in the subsequent determination of the cell density in the event of corresponding dosing liquid, the at least one cell density class is assigned, wherein the cell density of the dosing liquid is determined with this assigned at least one cell density class.

7. The method according to claim 6, wherein the processing apparatus is parameterized with a plurality of test liquids having different known cell densities, wherein a plurality of cell density classes is determined taking into account the cell density determined for each of the plurality of test liquids.

8. The method according to claim 6, wherein the different viscosity classes are determined using a cell concentration-based model for machine learning.

9. The method according claim 1, wherein the dosing apparatus comprises a second dosing liquid reservoir, the second dosing liquid reservoir being fluidically connected to the second fluid channel section, the dosing liquid flowing out of the second fluid channel section flowing into the second dosing liquid reservoir.

10. The method according to claim 9, wherein a difference of at least 60 mbar is present between a first internal pressure formed in the first dosing liquid reservoir and a second internal pressure formed in the second dosing liquid reservoir.

11. The method according claim 1, wherein the dosing apparatus is part of a bioreactor, wherein the dosing liquid further comprises a nutrient.

12. The method according to claim 11, wherein the dosing apparatus further comprises a nutrient sensor, wherein a nutrient concentration of the dosing liquid is determined with the nutrient sensor, wherein the processing apparatus additionally processes the nutrient concentration of the dosing liquid to determine the cell density of the dosing liquid.

13. The method according to claim 1, wherein the fluid channel pressure sensor is arranged in such a way that the fluid channel pressure sensor does not directly contact the sample liquid, wherein the pressure source is configured as a hydraulic pressure source and a hydraulic fluid is used as pressure fluid, wherein the first dosing liquid reservoir is pressurized from the outside with the pressure fluid by means of the pressure source.

14. The method according to claim 1, wherein the dosing apparatus further comprises a first reservoir pressure sensor associated with the first dosing liquid reservoir, wherein during the determination of the fluid pressure profile with the first reservoir pressure sensor, a first internal pressure formed in the first dosing liquid reservoir is determined, wherein the processing apparatus additionally processes the first internal pressure to determine the cell density of the dosing liquid.

15. A dosing apparatus comprising: a fluid channel, a valve, a pressure source, a first dosing liquid reservoir in which a dosing liquid can be accommodated, a processing apparatus, and a fluid channel pressure sensor associated with the fluid channel,
wherein the fluid channel comprises a first fluid channel section and a second fluid channel section fluidically connected to the first fluid channel section,
wherein the valve comprises a valve member that is arranged between the first and second fluid channel sections and can be moved into an open position, in which the fluidic connection of the first fluid channel section to the second fluid channel section is opened, and into a closed position, in which the fluidic connection of the first fluid channel section to the second fluid channel section is closed,
wherein the first dosing liquid reservoir is fluidically connected to the first fluid channel section,
wherein the dosing liquid comprises biological cells,
wherein the processing apparatus is configured to carry out a method comprising the steps of:
pressurizing the first dosing liquid reservoir with a pressurized fluid by means of the pressure source so that the dosing liquid is moved by pressurization in the first fluid channel section,
moving the valve member from the closed position to the open position at the beginning of a dosing time so that the dosing liquid is moved into the second fluid channel section and flows out of the second fluid channel section,
moving the valve member from the open position to the closed position at the end of the dosing time,
determining a fluid pressure curve during the dosing time and/or after the dosing time using the fluid channel pressure sensor,
wherein the processing apparatus processes the fluid pressure curve in order to determine a cell density of the dosing liquid.

16. The dosing apparatus according to claim 15, wherein the valve is configured as a pinch valve.

17. The dosing apparatus according to claim 15, wherein the valve is configured to be solenoid-operated.

18. A bioreactor comprising a dosing apparatus according to claim 15.
